(19) United States
(12) Patent Application Publication
(10) Pub. No.: US 2025/0082378 A1
(43) Pub. Date: Mar. 13, 2025

(54) FIBULAR NAIL OUTRIGGER SURGICAL DEVICE

(71) Applicant: Medartis AG, Basel (CH)

(72) Inventor: Kent KOHART, Warsaw, IN (US)

(73) Assignee: Medartis AG, Basel (CH)

(21) Appl. No.: 18/885,354

(22) Filed: Sep. 13, 2024

Related U.S. Application Data
(60) Provisional application No. 63/582,367, filed on Sep. 13, 2023.

(51) Int. Cl. A61B 17/74; A61B 17/72

(52) U.S. Cl. CPC A61B 17/744; A61B 17/7241

(57) ABSTRACT

An outrigger that aids the internal fixation of an intramedullary nail includes a nail attachment portion that attaches to an end of an intramedullary nail and a main outrigger body that attaches to the nail attachment portion. The main outrigger body includes a first portion with a first plurality of apertures that line up with a first plurality of apertures of the intramedullary nail and a second portion with a second plurality of apertures that line up with a second plurality of apertures of the intramedullary nail. The main outrigger body further includes a wire aperture, a connecting flange that connects the first portion and the second portion, and a mechanism that releasably locks the outrigger's apertures in alignment with the intramedullary nail's apertures.

### CROSS-REFERENCE TO RELATED APPLICATION

This application claims the benefit under 35 U.S.C. § 119(c) of U.S. Provisional Application No. 63/582,367 filed Sep. 13, 2023, the complete disclosure of which is hereby incorporated by reference in its entirety.

This application is related to application Ser. No. 17/996,308, filed on Oct. 14, 2022, entitled “Improved Linear Fibular Nail” (Attorney Docket No. 3768.116A), the entire disclosure of which is hereby incorporated by reference.

### TECHNICAL FIELD

The present invention relates to apparatuses, devices, and methods for aiding the reinforcement and healing of a fibula. More specifically, the present invention relates to apparatuses, devices, and methods for aiding internal fixation of a fibular nail.

### BACKGROUND OF THE INVENTION

The fibula is a non-weight bearing bone on the lower leg, connected to the tibia by ligaments such as the interosseous ligament, transverse tibiofibular ligament, anterior inferior tibiofibular ligament, and the posterior inferior tibiofibular ligament. These ligaments form a syndesmosis joint, a slightly movable joint that connects the fibula and tibia.

Fibulas may fracture or break, requiring surgery. Often, these surgeries require open reduction, where surgeons reposition the bone pieces so that the bones are in proper alignment, and internal fixation, where the bones are physically reconnected, often with special screws, plates, wires, nails, and/or other surgical fastening devices.

One such method of internal fixation requires drilling a cavity through a distal end of a fibula and inserting a fibular nail inside the fibula. From there, screws and wires are placed through the fibular nail. Some prior art fibular nails are rigid and have a curved tip at the proximal end of the fibular nail. The rigidity is meant to provide a stable structure for healing and the curved tip is designed to fit the curve of the fibula. However, these two features have drawbacks. The curved tip and rigidity can limit open reduction after the insertion of the fibular nail. It would be beneficial for a surgeon to have the ability to manipulate the placement of the bones both prior to and post insertion of the fibular nail.

In response to this need, a linear fibular nail that allows for open reduction of the fibula both prior to and post insertion of the fibular nail into the fibula, while still providing a stable structure for healing, is being developed, such as the Improved Linear Fibular Nail of co-owned application Ser. No. 17/996,308.

Internal fixation of a fibular nail can be complicated because the insertion of a fibular nail obscures the fibular nail from view, along with the fibular nail's cavities and slots designed to receive screws, wires, and other surgical fastening devices.

Thus, a need exists for improved systems and methods for internally fixating a bone to an intramedullary nail.

### SUMMARY OF THE INVENTION

The present invention provides an outrigger that aids the internal fixation of an intramedullary nail. The outrigger includes a nail attachment portion that attaches to an end of the intramedullary nail and a main outrigger body that attaches to the nail attachment portion. The main outrigger body includes a first portion with a first plurality of apertures that line up with a first plurality of apertures of the intramedullary nail and a second portion with a second plurality of apertures that line up with a second plurality of apertures of the intramedullary nail. The main outrigger body further includes a wire aperture, a connecting flange that connects the first portion and the second portion, and a mechanism that releasably locks the outrigger's apertures in alignment with the intramedullary nail's apertures.

The present invention further provides a method of internal fixation. The method includes inserting an intramedullary nail into a bone and attaching an outrigger to a base of the intramedullary nail. A first plurality of fasteners is fastened into a first plurality of cavities of the intramedullary nail by inserting the first plurality of fasteners through a first plurality of cavities of the outrigger. The first portion of the bone is positioned relative to a second portion of the bone. A surgical wire is inserted through a slot on the outrigger to hold the first portion of the bone or the second portion of the bone in place. A second plurality of fasteners are fastened into a second plurality of cavities of the intramedullary nail by inserting the second plurality of fasteners through a second plurality of cavities of the outrigger. The outrigger is removed from the base of the intramedullary nail.

### BRIEF DESCRIPTION OF THE DRAWINGS

FIG. 1 is a perspective view of an assembly of an embodiment of an outrigger attached to a fibular nail, showing the fibular nail inserted in a fibula;

FIG. 2 is a perspective view of the fibular nail of FIG. 1 inserted into the fibula;

FIG. 3 is a perspective view of the assembly of FIG. 1;

FIG. 4 is a partial perspective view of the assembly of FIG. 1, showing the outrigger's apertures in alignment with the fibular nail's apertures;

FIG. 5 is a partial perspective view of the assembly of FIG. 1, where the outrigger is releasably positioned in an unobstructed position;

FIG. 6 is a perspective view of an assembly of an alternate embodiment of an outrigger and the fibular nail of FIG. 1;

FIG. 7 is a partial side view of the assembly of FIG. 6;

FIG. 8 is a perspective view of the assembly of FIG. 6;

FIG. 9 is a front view of a releasably locking mechanism for hinging the outrigger of FIG. 6;

FIG. 10 is a front view of the releasably locking mechanism of FIG. 9 when the releasably locking mechanism is in a locked state;

FIG. 11 is a perspective view of the assembly of FIG. 6, showing a tibia and fibula, and a surgical wire inserted through a wire aperture in the outrigger and further through the fibula;

FIG. 12 is a perspective view of the assembly of FIG. 6, showing a tibia and fibula, and a surgical wire inserted through a wire slot in the outrigger and further through a tibia;

FIG. 13 is a perspective view of the assembly of FIG. 6, showing a tibia and fibula, with multiple surgical wires inserted through multiple cavities in the outrigger and further through the tibia and the fibula;

FIG. 14 is a first exploded assembly view of the outrigger of FIG. 6;

FIG. 15 is a second exploded assembly view of the outrigger of FIG. 6;

FIG. 16 is a rear perspective view of an embodiment of an outrigger where the outrigger is in a locked state;

FIG. 17 is a rear perspective view of the outrigger of FIG. 16 where the outrigger is in an unlocked state;

FIG. 18 is a front perspective view of the outrigger of FIG. 16 where the outrigger is in a locked state;

FIG. 19 is a front perspective view of the outrigger of FIG. 16 where the outrigger is in an unlocked state;

FIG. 20 is a perspective view of an assembly of an embodiment of an outrigger with the fibular nail of FIG. 1;

FIG. 21 is an exploded assembly perspective view of the assembly of FIG. 20;

FIG. 22 is a perspective view of an assembly of an embodiment of an outrigger with the fibular nail of FIG. 1;

FIG. 23 is an exploded assembly view of the assembly of FIG. 22;

FIG. 24 is a first exploded assembly view of an assembly of an alternate embodiment of an outrigger; and

FIG. 25 is a second exploded assembly view of the assembly of FIG. 24.

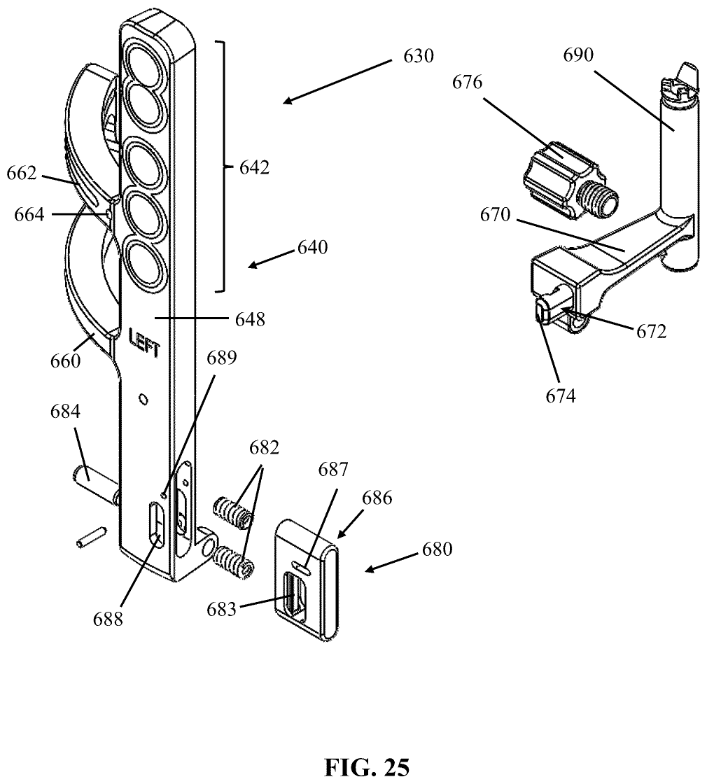

### DETAILED DESCRIPTION OF THE INVENTION

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

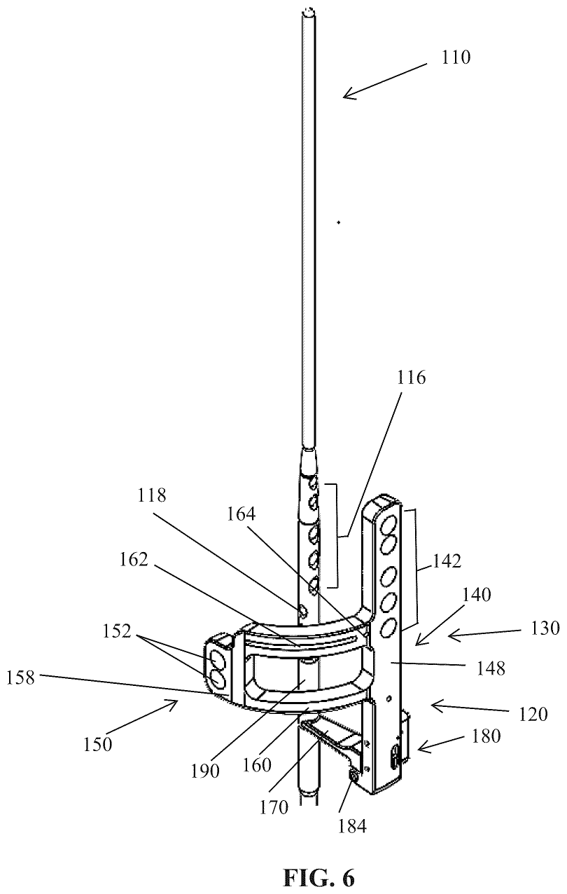

Thus, all the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “top”, “bottom”, “left”, “rear”, “right”, “front”, “vertical”, “horizontal”, “proximal”, “distal” and derivatives thereof shall relate to the invention as oriented in FIG. 6.

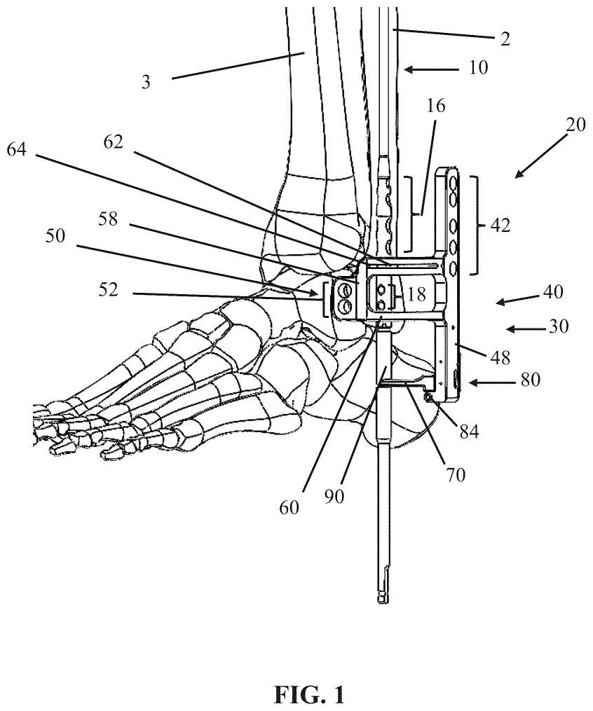

In accordance with the principles of the present invention, an outrigger that aids in fastening an intramedullary nail into a bone, e.g., a fibular nail into a fibula, is provided within. In an embodiment of the present invention depicted in FIGS. 6-15, an outrigger 120 may be attached to a fibular nail 110 that has been inserted into a fibula 2 (FIG. 1). While the fibular nail 110 may be inserted into the fibula 2, the outrigger 120 may be externally positioned from the fibula 2 and may be intended to be removed after the fibular nail 110 has been surgically fixed into the fibula. The fibular nail 110 may be identical or similar in one example to a linear fibular nail 30 described in co-owned application Ser. No. 17/996,308. As illustrated in FIGS. 6-15 of the present application, the outrigger 120 may include a main outrigger body 130, a nail attachment portion 190, and an attachment flange 170.

FIG. 6 depicts an embodiment of the outrigger 120. The outrigger 120 may be removed from the fibular nail 110 after insertion or fixation of the fibular nail 110 into the fibula 2 (FIG. 1). Alternatively, the fibular nail 110 may be inserted into the fibula 2 (FIG. 1) through other means (other than being manipulated while attached to the outrigger 120). The outrigger 120 thus may be attached to the fibular nail 110 after the fibular nail 110 has been inserted into the fibula 2 (FIG. 1) instead of before.

FIGS. 6-10 and 14-15 depict an embodiment of the outrigger 120 without the fibula 2 (FIG. 1) for simplicity of illustration. Referring to FIG. 6, the nail attachment portion 190 may releasably attach (e.g., via a threaded connection) to the fibular nail 110 at a distal end 112 of the fibular nail 110. A handle (not illustrated) may attach to the nail attachment portion 190. In the current embodiment, the nail attachment portion 190 may have an approximately cylindrical shape with grooves for a handle to attach at multiple points along a length of nail attachment portion 190. However, several shapes of the nail attachment portion 190 are contemplated. The attachment flange 170 of the outrigger 120 may connect the main outrigger body 130 to the nail attachment portion 190. In the current embodiment, the attachment flange 170 may have an approximately rectangular shape that extends between the nail attachment portion 190 and the main outrigger body 130. However, several shapes of the attachment flange 170 are contemplated.

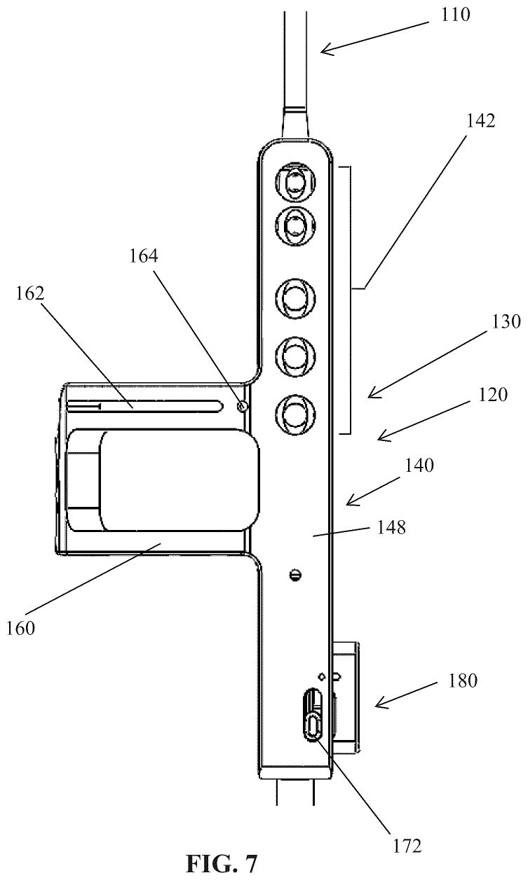

The main outrigger body 130 may include a first portion 140 that includes a plurality of first aligning apertures 142 at a top end 144 of the first portion 140. The first aligning apertures 142 may be configured to line up with and receive screws set to be inserted into a plurality of first nail apertures 116 of the fibular nail 110. FIG. 7 depicts a right-side view of the main outrigger body 130 with first aligning apertures 142 aligned with complementary first nail apertures 116 of a fibular nail 110. As illustrated in the differences in the apertures between the top two and the bottom three first nail apertures 116 of the fibular nail 110, some apertures may be configured to receive multiple and/or different types of screws than other apertures. Therefore, the first aligning apertures 142 may similarly be complementarily configured to receive the screws that the first nail apertures 116 are configured to receive and may deviate from the illustration depicted in FIGS. 6-8 and 11-15. Referring back to FIG. 6, the first portion 140 may have smoothed edges that may help a surgeon hold the outrigger 120. The top left and top right edges of the first portion 140 may have filleted edges.

The main outrigger body 130 may further include a second portion 150 that has a face 158 approximately perpendicular to a face 148 of the first portion 140. The second portion 150 may include a plurality of second aligning apertures 152. These second aligning apertures 152 may be configured to align with the second nail apertures 118 of the fibular nail 110 and to receive screws. Though the second nail apertures 118 of the fibular nail 110 are illustrated in FIGS. 6, 8, and 11-15 as having a consistent shape and depth, some aperture(s) of the second nail apertures 118 may be configured to receive multiple and/or different types of screws than other aperture(s) of the second nail apertures 118. Therefore, the second aligning apertures 152 may similarly be complementarily configured to receive the screws that the second nail apertures 116 are configured to receive and may deviate from the illustrations depicted in FIGS. 6, 8, and 11-15.

The face 158 may have a longitudinal axis and a transverse axis and the face 148 may have a longitudinal axis and a transverse axis. The longitudinal axis of the face 158 and the longitudinal axis of the face 148 may be approximately parallel to a longitudinal axis of the intramedullary nail (e.g., the fibular nail 110) such that screws may be received through the first aligning apertures 142 and the second aligning apertures 152 and into the first nail apertures 116 and the second nail apertures 118, respectively, at a desirable angle. Furthermore, the transverse axis of the face 148 and the transverse axis of the face 158 may be approximately perpendicular to each other to align with the first nail apertures 116 and the second nail apertures 118, respectively.

A connecting flange 160 may connect the first portion 140 and the second portion 150 as depicted at least in FIGS. 6-8. Connecting flange 160 may have a circumferential or arc shaped body with a radius about a longitudinal axis of the intramedullary nail (e.g., the fibular nail 110) and/or the nail attachment portion 190 when the intramedullary nail is connected to the outrigger 130 via the nail attachment portion 190 thereof. The connected flange 160 may be bounded by the approximately perpendicular face 148 of the first portion 140 and the face 158 of the second portion 150. However, other shapes of the connecting flange 160 are contemplated that connect the first portion 140 and the second portion 150. A height (e.g., in a direction parallel to the face 148 of the first portion 140) of the connecting flange 160 may be larger than a height (e.g., in a direction parallel to the face 158 of the second portion 150) of the second portion 150 and smaller than the height of the first portion 140. The bottom end of the second portion 150 and the bottom end of the connecting flange 160 may form one continuous line or surface. Faces 168 where the connecting flange 160 meets the first portion 140 and the second portion 150 may be filleted. The top end 144 of the first portion 140 may extend above the top end of the connecting flange 160 and the bottom end 146 of the first portion 140 may extend below the bottom end of the connecting flange 160.

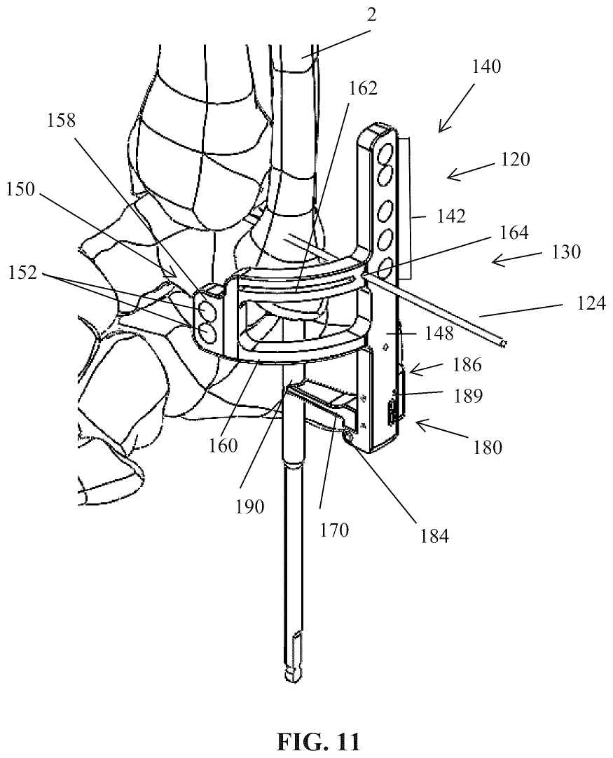

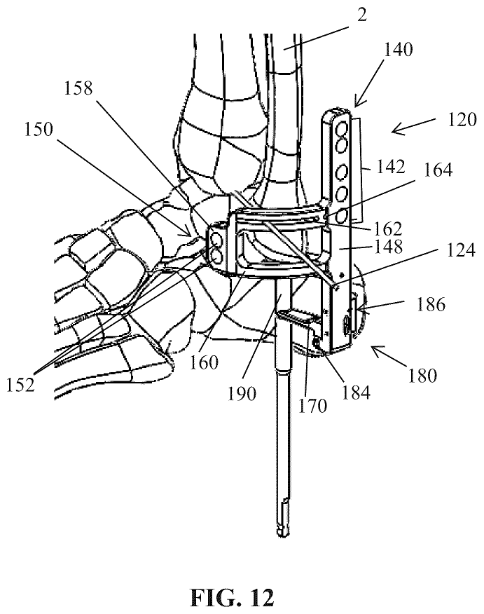

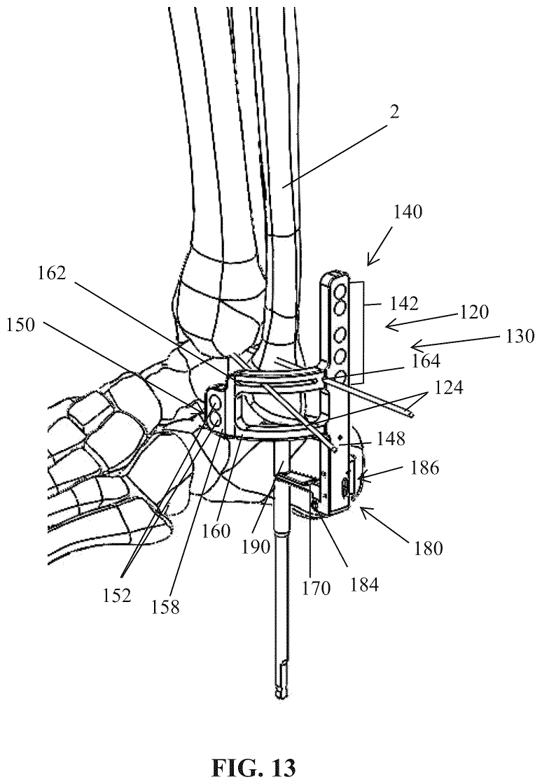

A wire slot 162 and/or a wire aperture 164 may be located on the connecting flange 160 and may be configured to receive a Kirschner wire 124 (or K-wire) (FIG. 13). The wire slot 162 may form a more elongated cavity than the wire aperture 164 to accommodate horizontal movement of a Kirschner wire 124 (FIG. 13). The wire slot 162 may extend through the connecting flange's 160 circumferential or arc shaped body, where the top and bottom edges of the wire slot 162 are longer than the left and right edges of the wire slot 162. The edges of the wire slot 162 may be filleted. The wire aperture 164 may be located on the connecting flange 160, approximately along a same horizontal axis as a horizontal axis of the wire slot 162 but may have a smaller width than a width of the wire slot 162 so as to provide a smoother locking fit for the Kirschner wire 124 (FIG. 12) to be placed through. As demonstrated in FIG. 6, the wire aperture 164 may be located to the right of the wire slot 162. The wire aperture 164 may be used to receive and lock the Kirschner wire 124 (FIG. 11) into place relative to the outrigger 120, while the wire slot 162 may be used to receive the Kirschner wire 124 (FIG. 11) while allowing the fibular nail 110 to be rotated. As seen in FIG. 6, the left and right bounds of wire slot 162 may define a maximum rotation of approximately ninety degrees with one continuous rotation of the Kirschner wire 124 (FIG. 12) when the Kirschner wire 124 is inserted through the wire slot 162 into a bone (the bone illustrated in FIGS. 12-13). However, the illustration of wire slot 162 should not be construed as limiting, as the length of wire slot 162 may be shortened or lengthened and thus the total degree and angle of rotation of the Kirschner wire 124 through the wire slot 162 may be lessened, expanded, and/or adjusted. The wire aperture 164 may be used lock the fibula 2 and/or the fibular nail 110 into place with the second Kirschner wire 124 (FIG. 13) or other surgical wire, which may provide additional fixation of the fibula 2, after the first placed Kirschner wire 124 (FIG. 13) was used to rotate the fibula 2 and/or the fibular nail 110. After the second Kirschner wire 124 (FIG. 13) may be locked into place with the fibular nail 110, the first and the second Kirschner wires 124 (FIG. 13) may be removed when a surgeon deems the fixation is no longer needed.

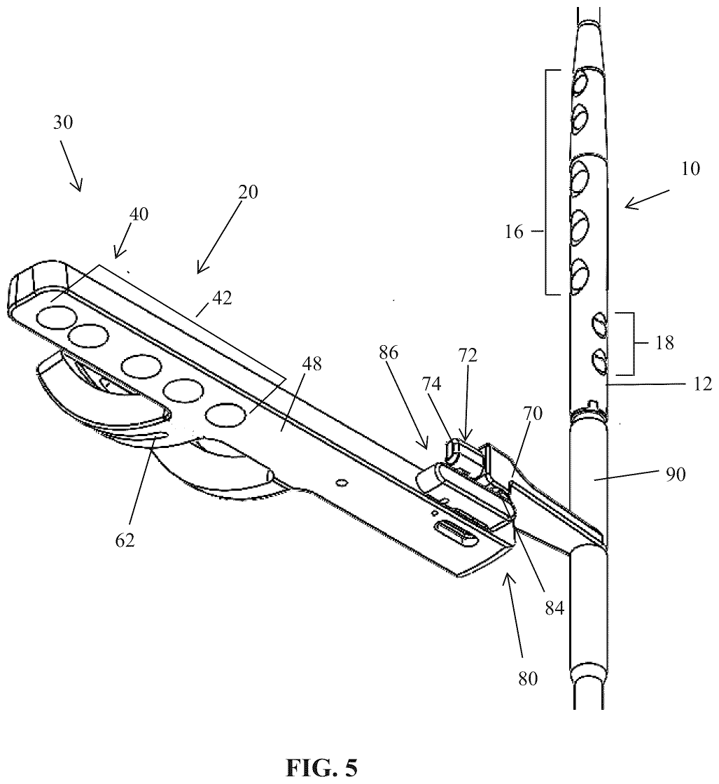

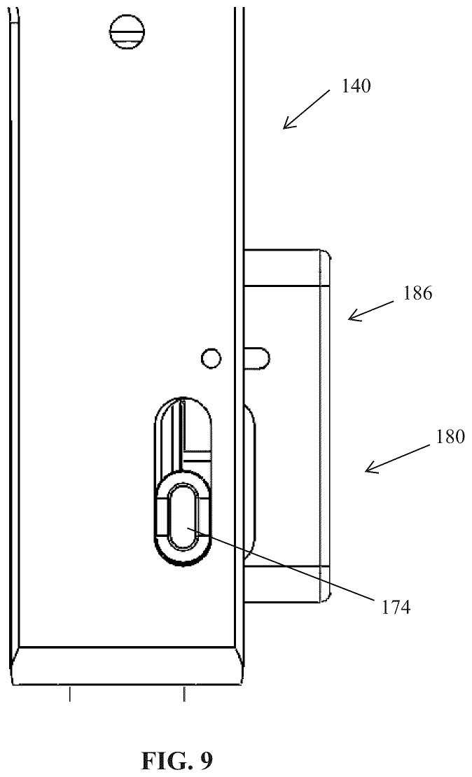

Furthermore, a releasably locking mechanism 180 may be located on the main outrigger body 130. In one embodiment, the releasably locking mechanism 180 may be located on a bottom end 146 of the first portion 140. Referring to FIGS. 9, 10, 14, and 15, the releasably locking mechanism 180 may include a button (i.e., a button 186) that actuates the release of the releasably locking mechanism 180 by pressing the button 186; a retention pin (not illustrated) that retains the button 186 in the releasably locking mechanism 180 and prevents the button 186 from falling outside the releasably locking mechanism 180; internal springs (e.g., internal springs 182) that aid the actuation of button 186 and/or biases the return of button 186; and a tapered surface 183, a tapered cutout 172, and an internal cavity 188 that aid the internal workings of the releasably locking mechanism 180. The releasably locking mechanism 180 may operate by pressing the button 186, which may shift button 186 further into cavity 188 in the first portion 140 to allow a movement of the main outrigger body 130 to an unobstructed position (FIG. 5).

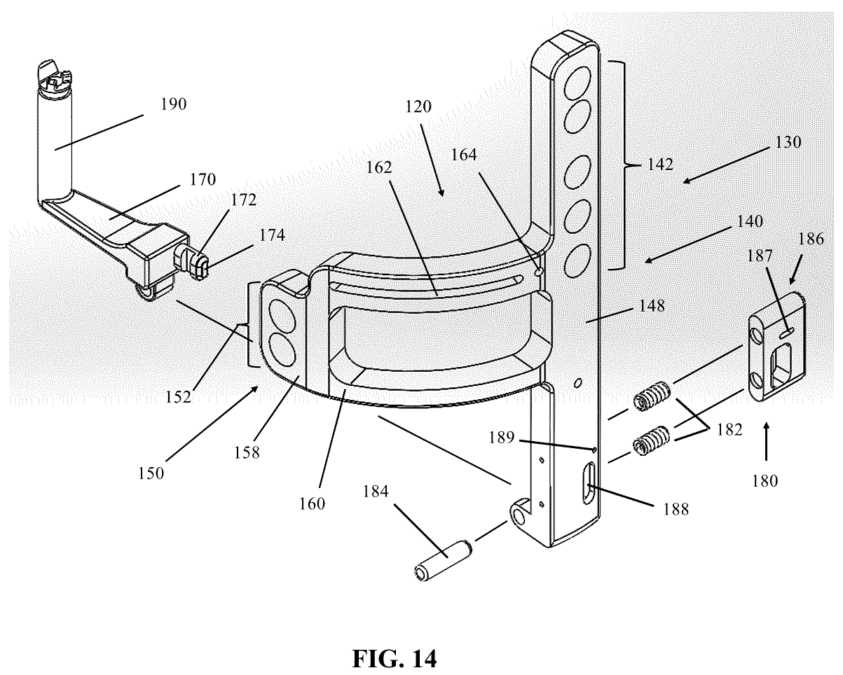

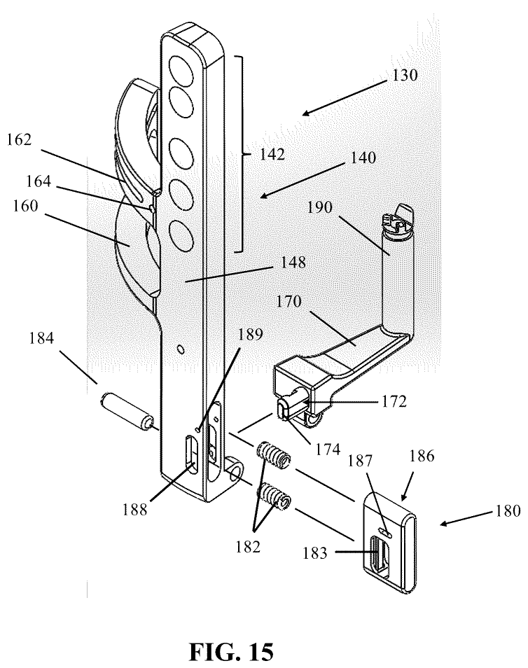

To assemble the releasably locking mechanism 180, the internal springs 182, which may have ends 181, may be inserted into and connected to a surface bounding the internal cavity 188, as depicted for example in FIGS. 14-15. Furthermore, the button 186 may be inserted within the internal cavity 188 and be placed in contact with and/or over a top of the internal springs 182 near the bottom end 146 surface of the first portion 140 (FIG. 6). The rotation pin 184 (FIGS. 14 and 15) may be located between the main outrigger body 130 and attachment flange 170, around which the main outrigger body 130 may rotate. The retention pin (not illustrated) may be inserted into a small hole 189 located above the internal cavity 188 and through the slot 187 in the button 186 to secure the internal springs 182 and the button 186 inside of the main outrigger body 130.

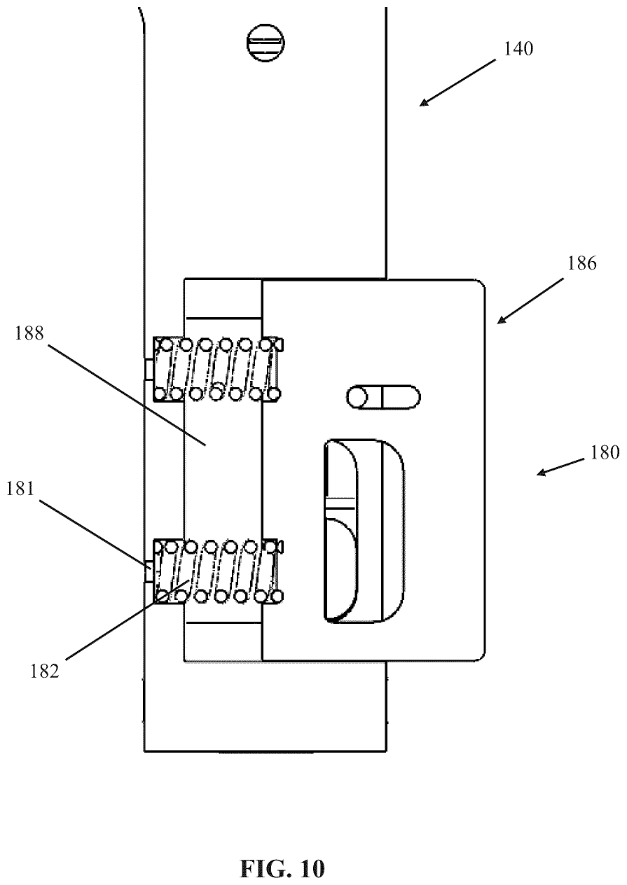

Referring to FIG. 6, to operate releasably locking mechanism 180, the button 186 (FIG. 10) may be pressed to compress the internal springs 182 (FIG. 10). When the button 186 (FIG. 10) is not pressed, such that springs 182 are in an uncompressed state, the outrigger 120 may remain locked in a presently depicted alignment position, using a tapered surface 183 on the button 186 (FIG. 10) opposing a tapered cutout 172 (FIGS. 14 and 15) on the portion extending through an internal cavity 188 of the first portion 140. When the button 186 (FIG. 10) is pressed, the tapered surface 183 and the tapered cutout 172 (FIGS. 14 and 15) may disengage and allow the main outrigger body 130 to rotate around the rotation pin 184 after any Kirschner wires 124 are removed from any bones (e.g., the fibula 2) and the wire slot 162 and the wire aperture 164. After the main outrigger body 130 is moved from its alignment position (i.e., alignment position demonstrated in FIG. 6), the tapered surface 183 and the tapered cutout 172 (FIGS. 14 and 15) may no longer be engaged, allowing the main outrigger body 130 to rotate around the rotation pin 184 (FIGS. 14 and 15) without engaging the releasably locking mechanism 180 into an unobstructed position (similar to an unobstructed position of the outrigger 20 depicted in FIG. 5), allowing an unobstructed view of the surgery site. In this unobstructed position, a surgeon may visualize what the injury site will look like with the outrigger 120 detached from the fibular nail 110, without having to fully detach the fibular nail 110 from the outrigger 120. This may simplify the number of steps or processes a surgeon must make, resulting in better patient outcomes. To lock the main outrigger body 130 back into the alignment position, the main outrigger body 130 may be manually moved back into the alignment position with or without pressing the button 186 because a taper 174 (FIGS. 14 and 15) on the tapered cutout 172 (FIGS. 14 and 15) extending from the internal cavity 188 allows the button 186 (FIG. 10) to automatically be pressed and released, which allows the main outrigger body 130 to rotate into the alignment position and the button 186 (FIG. 10) to engage and lock. After the screws have been inserted into the first nail apertures 116/second nail apertures 118 of the fibular nail 110 and the intermedullary bone (e.g., the fibula 2), and the surgery has been positively evaluated, the nail attachment portion 190 may be detached from the fibular nail 110. The main outrigger body 130 and the attachment flange 170 may thereby also be removed, i.e., along with the detachment of the nail attachment portion 190.

After the outrigger 120 has been removed from the fibular nail 110, the internal fixation of the fibula 2 (FIG. 1) as described above may allow the fibula 2 (FIGS. 11-13) to heal. Several progressions of gradually increased loading to the leg may be used in order to avoid/limit weight bearing to the fibula 2 (FIGS. 11-13), such as a cast, a walking boot, a walking shoe, a pair of crutches, a knee scooter, a cane, etc.

FIG. 6 illustrates the main outrigger body 130 in an aligned position (also demonstrated in FIGS. 7-8 and 11-13) where the first aligning apertures 142 and the first nail apertures 116 of a fibular nail 110, as well as the plurality of second aligning apertures 152 and the second nail apertures 118 of the fibular nail 110 are concentrically aligned. In this position, a surgeon may rely on the location and position of the first aligning apertures 142 and the second aligning apertures 152 of the outrigger 120 apertures to set screws through the first aligning apertures 142 and the second aligning apertures 152 of the outrigger 120 and into the first nail apertures 116 and the second nail apertures 118 of the fibular nail 110. The depiction of the outrigger 20 in FIG. 5 contrasts this aligned position in an identical fashion to outrigger 120, showing a main outrigger body 30 in an unobstructed position in an identical position to how main outrigger body 130 would be positioned in an unobstructed position. Referring back to FIG. 6, the outrigger 120 moves into this unobstructed position after pressing the button 186 of the releasably locking mechanism 180 to release the releasably locking mechanism 180 (e.g., the connection between the attachment flange 170 and the first portion 140) as described above.

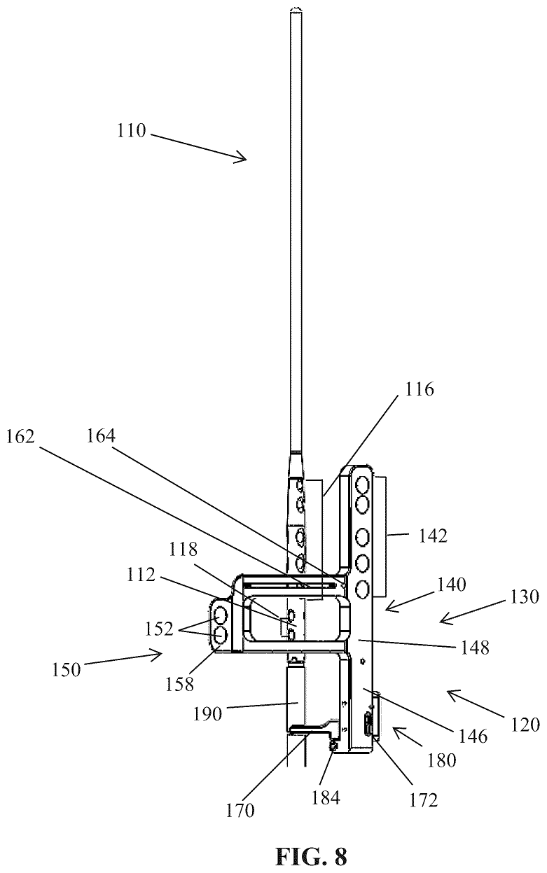

FIGS. 14-15 illustrate one way in which the main outrigger body 130 may be assembled. Multiple internal springs 182, for example two internal springs as illustrated, may be inserted into a releasably locking mechanism 180. In order to maintain the connection between the internal springs 182 and the releasably locking mechanism 180, the internal springs 182 may be connected to an internal cavity of the main outrigger body 130 through means such as glue, wherein the internal cavity of the main outrigger body 130 is located at a distal end 112 (FIG. 8) of the main outrigger body 130. The releasably locking mechanism 180 and the internal springs 182 may then be inserted into the internal cavity 188. Similarly, the connection between the internal springs 182 and the internal cavity 188 of the main outrigger body 130 may be maintained through means such as glue. Furthermore, the attachment flange 170 and the nail attachment portion 190 may be connected and the attachment flange 170 may contain the tapered cutout 172 and the taper 174. The button 186 (along with the attachment flange 170 and the nail attachment portion 190) may be inserted through the releasably locking mechanism 180 and the attachment flange 170 may be connected to the distal end 112 (FIG. 8) of the main outrigger body 130 through means such as a rotation pin 184 (FIGS. 14 and 15). The rotation pin 184 (FIGS. 14 and 15) may connect the attachment flange 170 to the main outrigger body 130 through means such as a press fit, glue, etc. The button 186 may facilitate the releasably locking mechanism 180.

In an embodiment of the present invention depicted in FIGS. 1 and 3-5, all of the features are nearly identical to the examples depicted in FIGS. 6-15 with nearly identical features offset by 100. For example, an embodiment of the outrigger 20 depicted in FIGS. 1 and 3-5 is nearly identical to an embodiment of the outrigger 120 depicted in FIGS. 6-15. However, the outrigger 20 may have a wire aperture 64, which may be alternatively located to the left of a wire slot 62, as opposed to the wire aperture 164 being located to the right of a wire slot 162 as depicted in FIGS. 6-8 and 11-15. The wire aperture 64 may be located on a connecting flange 60 in between a first portion 40 and a second portion 50, approximately along a same horizontal axis as a central horizontal axis of the wire slot 62.

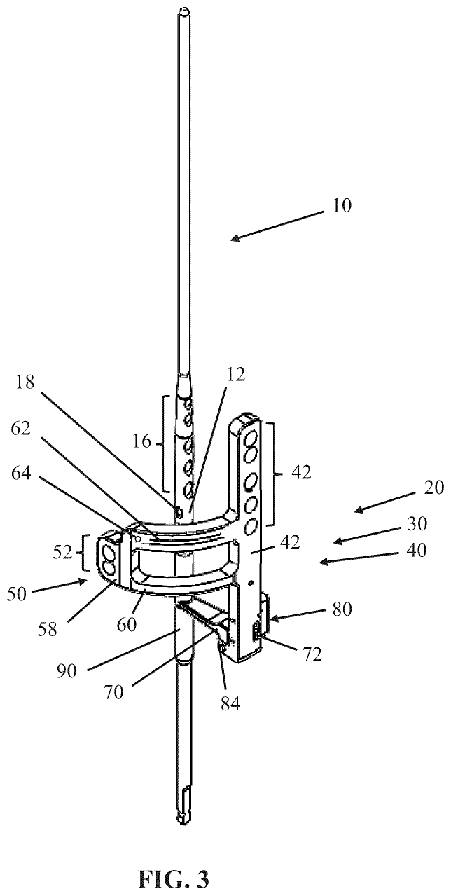

Referring to FIG. 3, a nail attachment portion 90 may releasably attach (e.g., via a threaded connection) to a fibular nail 10 at a distal end 12 of the fibular nail 10. An attachment flange 70 of the outrigger 20 may connect a main outrigger body 30 to the nail attachment portion 90. The nail attachment portion 90 may releasably attach (e.g., via a threaded connection) to the fibular nail 10 at a distal end 12 (FIG. 5) of the fibular nail 10. The main outrigger body 30 may have a first portion 40 with a plurality of first aligning apertures 42 at a top end 44 of a first portion 40. The first aligning apertures 42 may be configured to line up with and receive screws set to be inserted into first nail apertures 16 of fibular nail 10. Alternatively, the first aligning apertures 42 may be designed to receive suture buttons through the plurality of first nail apertures 16 of the fibular nail 10. The first portion 40 may form an approximately perpendicular body with smoothed edges. The top left and top right of the first portion 40 may have filleted edges. The releasably locking mechanism 80 of the outrigger 20 located at a bottom end 46 of the first portion 40 may be similar or identical to the releasably locking mechanism 180 of the outrigger 120 (FIGS. 6-15).

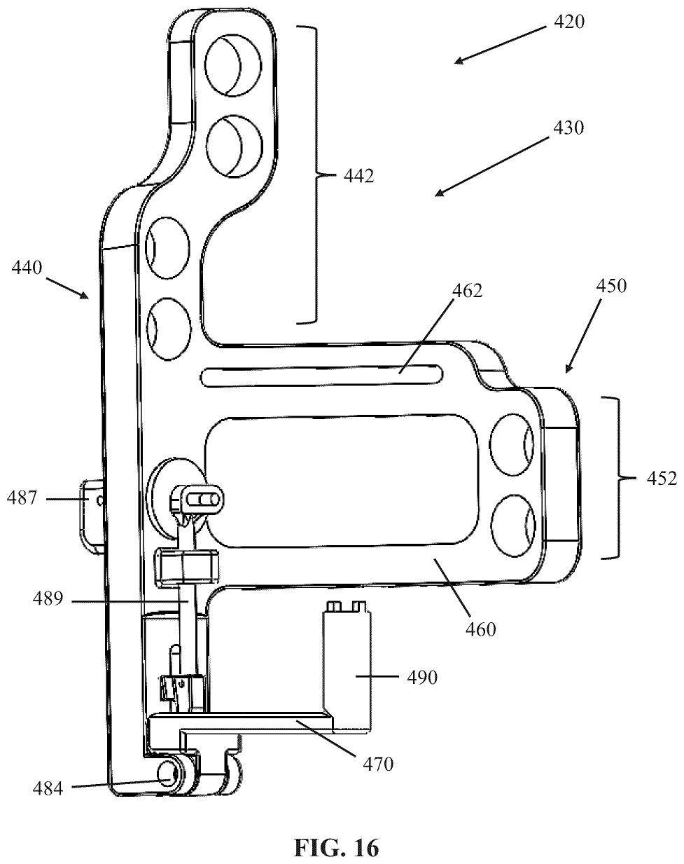

In another embodiment of the present invention, an outrigger 420 is depicted in FIGS. 16-19. The outrigger 420 may have similar features to the embodiment of the outrigger 120 with similar features offset by 300. Some of the main differences between the outrigger 420 and the outrigger 20 and the outrigger 120 described above include different releasably locking mechanisms (i.e., a releasably locking mechanism 480) and a differently shaped first portion (i.e., a first portion 440). Specifically, the first portion 440 of the outrigger 420 may have a plurality of first aligning apertures 442 with centers that do not align on the same plane, unlike the outrigger 120 that has a plurality of first aligning apertures 142 with centers that align on the same plane (illustrated in FIG. 6). The first portion 440 may have a non-linear shape for different iterations of a fibular nail (e.g., those having a different shape relative to fibular nail 210).

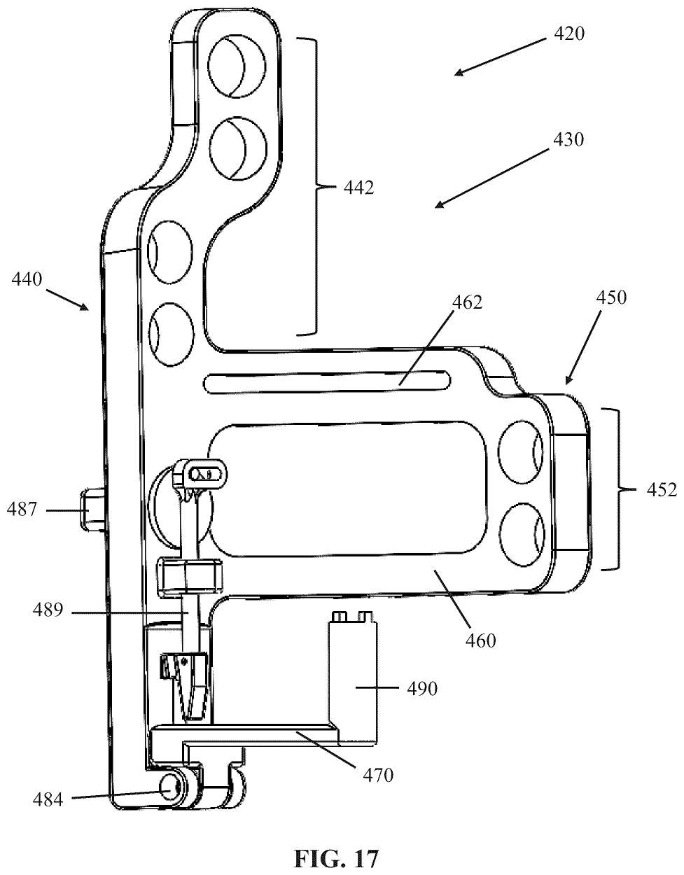

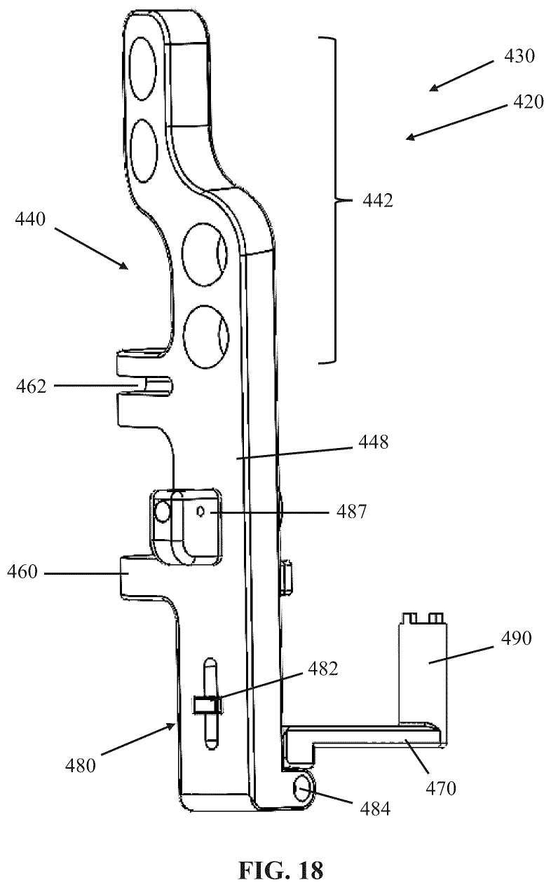

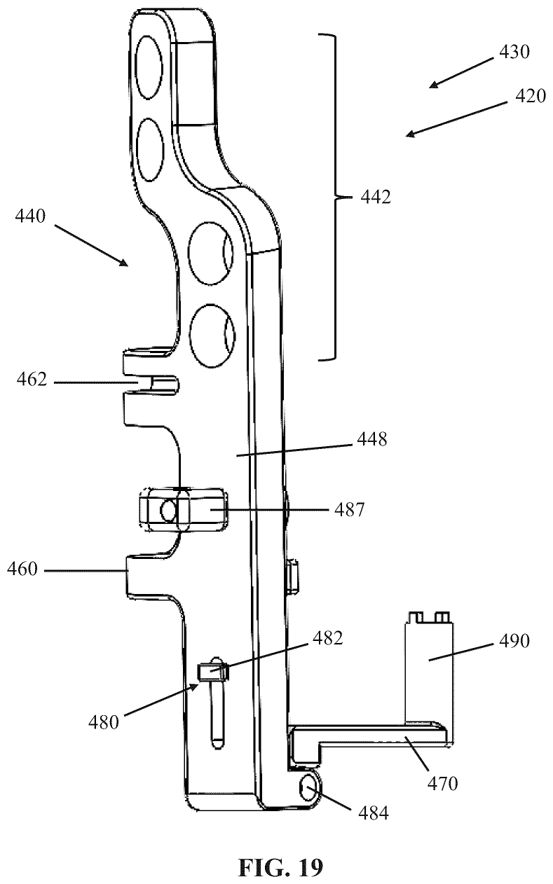

An actuation and release of releasably locking mechanism 480 illustrated in FIG. 18 and FIG. 19 may be actuated by a turn knob 487 that turns a locking mechanism 489. As is best seen in FIG. 16 and FIG. 18, a turn knob 487 may have a first locked position that keeps the main outrigger body 430 in line with an attachment flange 470. Rotating the turn knob 487 either manually or through other means to an unlocked position (illustrated in FIG. 17 and FIG. 19), may release a lock between the locking mechanism 489 and the attachment flange 470, thus allowing a main outrigger body 420 to releasably rotate from the attachment flange 470. A slider 482 attached to releasably locking mechanism 480 may illustrate and possibly aid the locking mechanism 487 (e.g., by limiting the maximum rotation of turn knob 487). As illustrated in FIG. 16 and FIG. 18, a slider 482 positioned centrally in a cavity may illustrate that the locking mechanism 489 is locked; while, as illustrated in FIG. 17 and FIG. 19, a slider 482 positioned in a higher end may illustrate that the locking mechanism 489 is unlocked. Any iteration or omission of the slider 482 that can illustrate and/or aid the release and/or locking of locking mechanism 489 may be used. Additionally, the outrigger 420 may or may not include a wire aperture (e.g., the wire aperture 64/164).

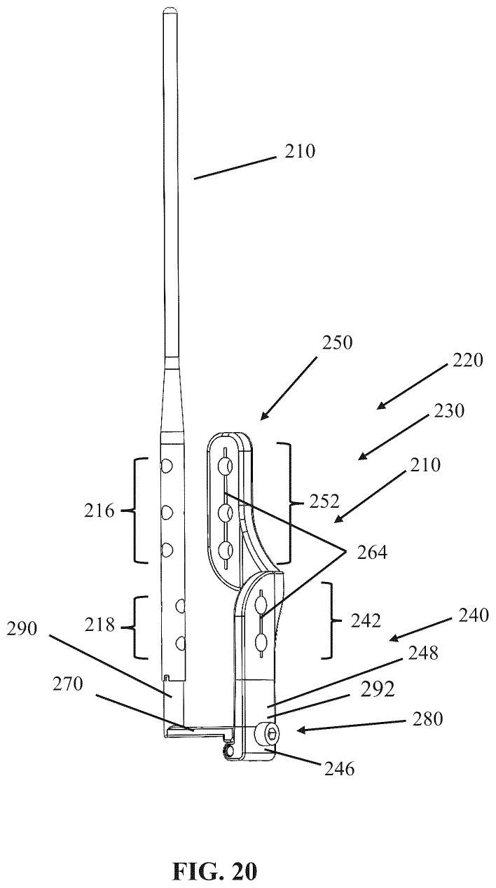

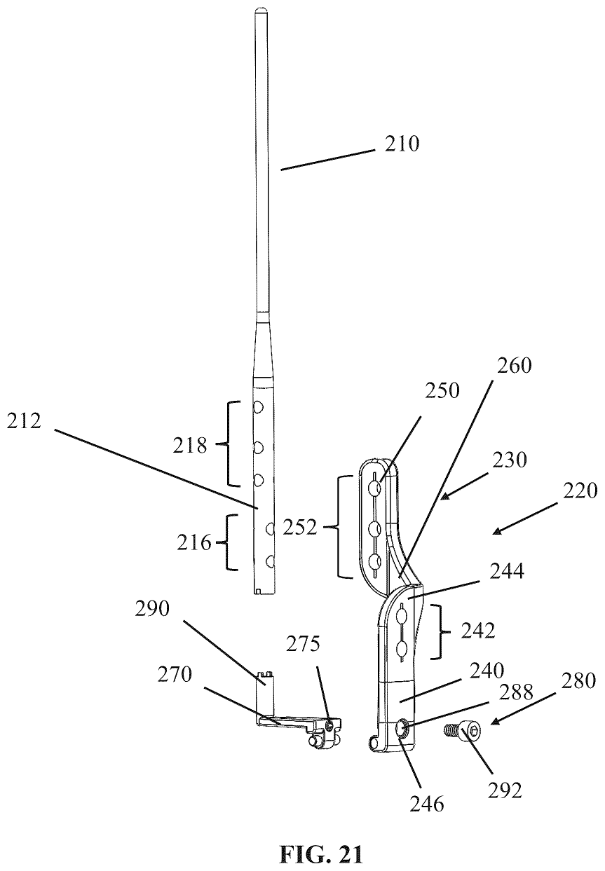

Another embodiment of the present invention is depicted in FIGS. 20-21. The main differences between this embodiment of an outrigger 220 and the previously described embodiments (i.e., outrigger 20/120/420) described above include a differently shaped connecting flange (i.e., a connecting flange 260), a releasably locking mechanism 280 actuated by a thumb screw 292, and incision apertures 264 located on a first elongated element 240 and a second elongated element 250. Outrigger 220 may include features similar to above described features of other embodiments of the outrigger, such as the outrigger 120, with reference numbers for such features offset by 100. In general, this embodiment includes a nail attachment portion 290 that may releasably attach (e.g., via a threaded connection) to a fibular nail 210 at a distal end 212 of the fibular nail 210. An attachment flange 270 of the outrigger 220 may connect a main outrigger body 230 to the nail attachment portion 290. The main outrigger body 230 may have the first portion 240 with a plurality of second aligning apertures 252 at a top end 244 of the first portion 240. The second aligning apertures 252 may be configured to line up with and receive screws set to be inserted into second aligning apertures 218 of the fibular nail 210. Alternatively, the second aligning apertures 252 may be designed to receive suture buttons through the plurality of second nail apertures 218 of the fibular nail 210. The first portion 240 may form an approximately perpendicular body with smoothed edges. The top left, top right, and bottom right edges of the first portion 240 may be filleted.

The main outrigger body 230 also may have a second portion 250 having an axis approximately perpendicular to an axis of the first portion 240. The second portion 250 may include a plurality of first aligning apertures 242. These first aligning apertures 242 may be configured to line up with and receive screws (or suture buttons) configured to be inserted into first aligning apertures 216 of the fibular nail 210. The top left edge of the second portion 244 may be filleted.

The connecting flange 260 may connect the first portion 240 and the second portion 250. The connecting flange 260 may have a circumferential or arc shaped body bounded by the approximately perpendicular first portion 240 and second portion 250. The height of the connecting flange 260 may be smaller than the height of the second portion 250 and even with the height of the first portion 240. The bottom end of the second portion 250 and the top end of the connecting flange 260 may form one continuous line or surface. The edges of the body where the connecting flange 260 meets the first portion 240 and the second portion 250 may have filleted edges.

Incision apertures 264 may be connected to the second aligning apertures 252 and/or the first aligning apertures 242 and may be configured for surgical blades to create incision to prepare for fixation (e.g., screws). The incision apertures 264 may form vertical apertures that approximately bisect the second aligning apertures 252 and/or the first aligning apertures 242.

Furthermore, the releasably locking mechanism 280 may be located on the main outrigger body 230, on the bottom end 246 of the first portion 240. The releasably locking mechanism 280 may releasably lock first portion 240, the second portion 250, and thus the first aligning apertures 242 and the second aligning apertures 252 into alignment with the first nail apertures 216 and the second nail apertures 218 of the fibular nail 210. When the main outrigger body 230 is in an alignment position (depicted in FIG. 20), the thumb screw 292 is inserted through a cavity 288 at a bottom end 246 of the first portion 240 into a threaded hole 275 on the attachment flange 270. The releasably locking mechanism 280 may release a connection between the attachment flange 270 and the first portion 240 to allow a rotation or movement of the main outrigger body 230 into an unobstructed position (similar to the unobstructed position of outrigger 130 depicted in FIG. 5), thereby allowing an unobstructed view of the surgery site. This connection may be released by unscrewing or removing the thumb screw 292 from the cavity 288/the threaded hole 275. In this unobstructed position, a surgeon will be able to visualize what the injury site will look like with the outrigger 220 detached from the fibular nail 210, without having to fully detach the fibular nail 210. The main outrigger body 230 may be manually positioned back into an alignment position, i.e., such that the first aligning apertures 242 and the second aligning apertures 252 are aligned with the first nail apertures 216 and the second nail apertures 218 of the fibular nail 210. After the screws have been inserted into the fibular nail 210 and the surgery has been evaluated, the nail attachment portion 290 may be detached from the fibular nail 210, and the main outrigger body 230 and the attachment flange 270 may also be removed, along with the detachment of the nail attachment portion 290.

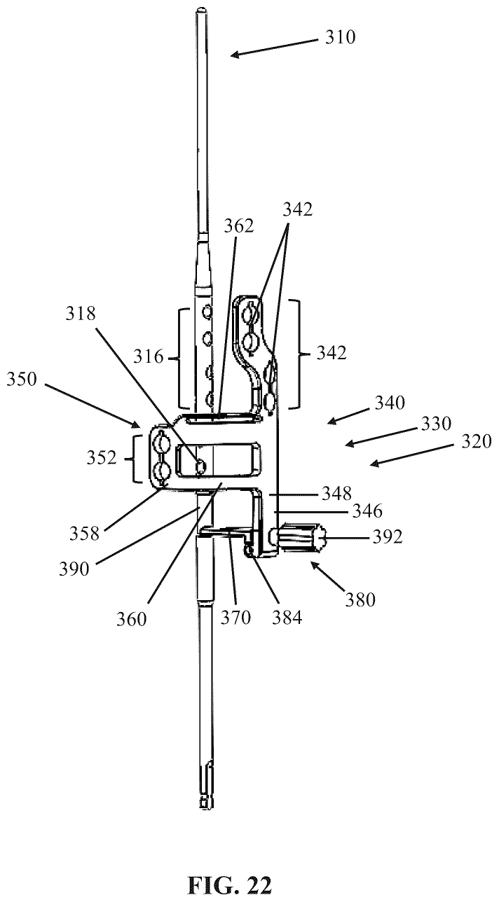

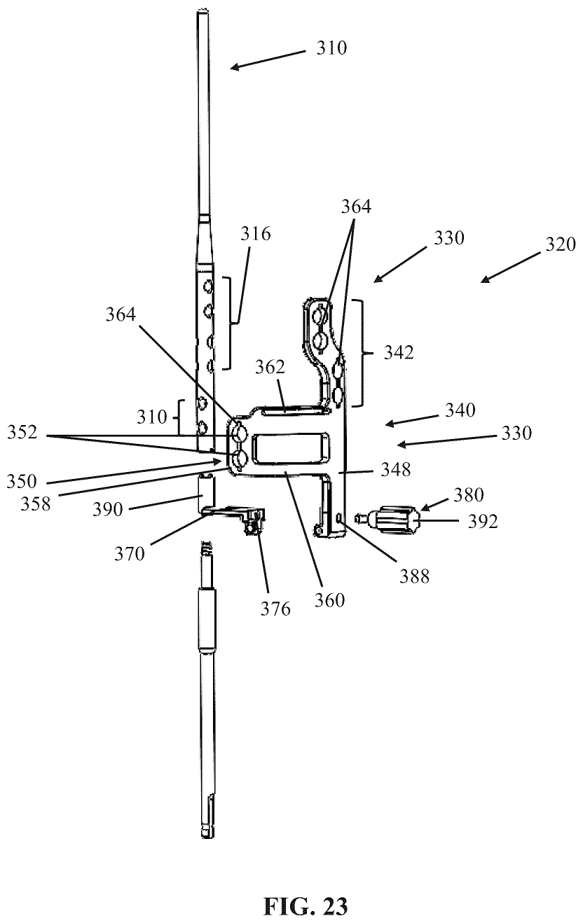

Another embodiment of the present invention is depicted in FIGS. 22-23. This embodiment of an outrigger 320 may include features of the previous embodiments (i.e., the outrigger 120/220/420) described above. This embodiment of the outrigger 320 may include features similar to above described features of other embodiments of the outrigger, such as outrigger 120, with reference numbers for such features offset by 200. For example, the outrigger 320 may have a main outrigger body 330 with a first portion 340, a connecting flange 360, and a second portion 350 with a nearly identical shape to the outrigger body's 420 first portion 440, connecting flange 460, and second portion 450. Multiple incision apertures 364 may approximately bisect the multiple apertures on the first portion 340 and the second portion 350, similar to the shape of the incision apertures 264 (FIG. 20). The outrigger 320 also contains a connecting flange 360 with a wire slot 362 similar to the wire slot 162 (FIGS. 6-8). Finally, the releasably locking mechanism 380 may be actuated by a key 392 through cavity 388 located at a distal end 346 of first portion 340 and into an undercut hole 376 in attachment flange 370, similar to the thumb screw 292 of outrigger 220 (FIG. 20), wherein the insertion and release of the key 392 in distally located cavity 388 on first portion 346/undercut hole 376 allows the main outrigger body 330 to rotate from an aligned position (depicted in FIG. 22) to an unobstructed position (depicted in FIG. 5).

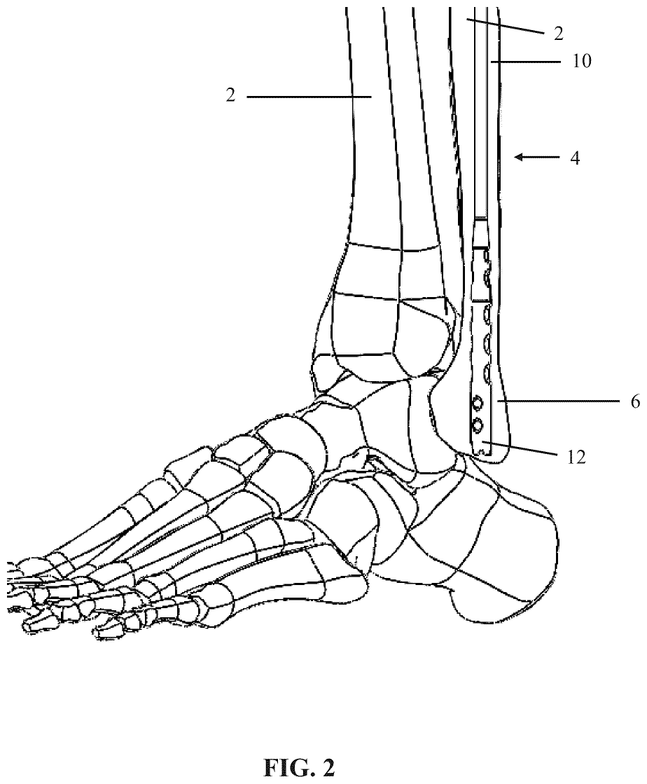

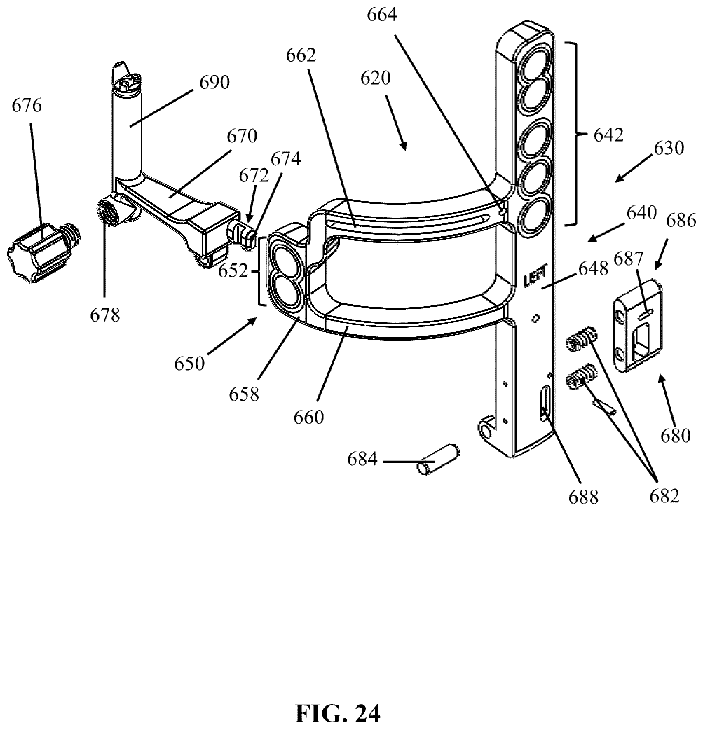

Another embodiment of the present invention is depicted in FIGS. 23-24. This embodiment of an outrigger 620 may include features of the previous embodiments (i.e., the outrigger 120) described above. This embodiment of the outrigger 620 may include features similar to the above described features of other embodiments of the outrigger, such as outrigger 120, with reference numbers for such features offset by 500. For example, the outrigger 620 may have a main outrigger body 630 with a first portion 640, a connecting flange 660, and a second portion 650 with a nearly identical shape to the outrigger body's 120 first portion 140, connecting flange 160, and second portion 150. The outrigger 620 may have a thumb screw 676 that may be inserted/screwed/fastened into a cavity 678, located on the attachment flange 670. The thumb screw 676 may assist in maintaining a connection between the nail connecting portion 690 and the attachment flange 670 (e.g., by providing a friction fit between the nail connecting portion 690 and the attachment flange 670). For example, during surgery, the outrigger 620 may experience movement in multiple directions, such as through insertion of screws, insertion of K-wires 124 (FIGS. 11-13), other intentional uses of the outrigger 620, and other unintentional interactions with the outrigger 620. The thumb screw 676 may limit a force or a movement applied to the nail connecting portion 690, thus retaining a connection between the nail connecting portion 690 and the intramedullary nail (e.g., the fibular nail 610) (not illustrated). The thumb screw 676 may provide additional fixation to attachment flange 670, the nail connecting portion 690, a connection between the attachment flange 670 and the nail connecting portion 690, and/or a connection between the nail connecting portion 690 and the intramedullary nail (e.g., the fibular nail 610) (not illustrated). The thumb screw 676 may prevent the intramedullary nail (e.g., the fibular nail 610) from being disengaged and provide additional stability in the event of pressure or rotational pressure applied from movement to the intramedullary nail, movement from a bone (e.g., the fibula 2 or the tibia 3 illustrated in FIGS. 1-2), and/or movement from screws inserted into the intramedullary nail.

A method of using the outrigger 20/120/220/320/420 (depicted in FIGS. 3, 6, 16, 20, and 22) is described below. After the fibular nail 10/110/210/310 has already been inserted into the fibula 2 (e.g., as described in co-owned U.S. application Ser. No. 17/996,308 incorporated by reference above and as depicted in FIG. 1), interlocking screws (not illustrated) may be drilled, inserted, or screwed through the second aligning apertures 52/152/252/352/452 and through the second nail apertures 18/118/218/318/418 of the fibular nail 10/110/210/310, at a location below a fracture line of fibula 2 (FIG. 1). These anterior to posterior interlocking screws (not illustrated) may be used to grab or pull a lowered fractured portion into alignment with the top fractured portion of the fibula 2 (FIG. 1) and/or to a particular fibula length as determined by a surgeon.

After the lower portion of the fibula 2 (FIG. 1) has been secured with screws (not illustrated), the first Kirschner wire 124 (FIG. 12) may be inserted through the wire slot 62/162/462 and into the fibular nail 10/110 to hold the position and placement of an upper part 4 of the fibula 2 and the lower part 6 of the fibula in a particular location or position relative to each other as determined by a surgeon (FIG. 1). Then, a pressure applied to the outrigger 20/120/220/320/420 or a handle (not illustrated) attached to nail attachment portion 90/190/290/390/490 may rotate the fibular nail 10/110/210/310. Alternatively, such pressure may be applied via a Kirschner wire without the aid of a wire slot (e.g., wire slot 62/162/462), e.g., when utilizing outrigger 220 which does not have a designated wire slot (e.g., wire slot 62/162/462). In other designs, this rotation may not be possible as the tip of an alternate design fibular nail may be curved, thus limiting rotation of the alternate design fibular nail because the curvature of the alternate design fibular nails intended to match the curvature of the fibula 2 would be incompatible with the fibula 2 if the alternate design fibular nails are rotated to a relatively significant degree. In one embodiment, the fibula 2 (FIG. 1) may be rotated five to ten degrees to align the fibula 2 (FIG. 1) in a position for healing by applying pressure to the first Kirschner wire 124 (FIG. 12) that has been placed into the fibula 2 (FIG. 1) through the wire slot 62/162/462. The fibula 2 (FIG. 1) may be rotated any number of degrees based on the nature and severity of the injury.

After the fibula 2 (FIG. 1) has been rotated to a surgeon's satisfaction, the second Kirschner wire 124 (FIG. 11 and FIG. 13) may be inserted through the wire aperture 64/164 and into the fibula 2, thus locking the fibula 2 (FIG. 1) into place. Then, the first Kirschner wire 124 (FIG. 13) and the second Kirschner wire 124 (FIG. 13) may be removed from the wire slot 62/162/462 and/or from the fibular nail 10/110/210/310 when a surgeon determines that the Kirschner wires 124 (FIG. 13) may be removed. Further, any type of surgical wire or bone fixation device may be used instead of the Kirschner wire 124. Additionally, the description of the use of two Kirschner wires 124 should not be construed as limiting as any number of Kirschner wires, surgical wires, or bone fixation devices may be used.

After the second Kirschner wire 124 (FIG. 13) may have provided additional fixation to the fibula 2 (FIG. 1), screws (not illustrated) may be screwed/drilled/inserted through the bottom first aligning apertures 42/142/242/342/442, through the bottom first nail apertures 16/116/216/316/416 of a fibular nail 10/110/210/310, and through soft tissue into a tibia 3 to help with damage incurred to soft issue. Then, screws (not illustrated) may be screwed/drilled/inserted through the remaining upper first aligning apertures 42/142/242/342/442, and into the upper first nail apertures 16/116/216/316/416 of a fibular nail 10/110/210/310.

To screw, drill, or insert an interlocking screw (not illustrated) and/or a suture button (not illustrated) through the first aligning apertures 42/142/242/342/442 and into the first nail apertures 16/116/216/316/416 of the fibular nail 10/110/210/310 or through the second aligning apertures 52/152/252/352/452 and into the second nail apertures 18/118/218/318/418 of the fibular nail 10/110/210/310, the following steps may be used. First, a drill guide (not illustrated) and a drill guide sleeve (not illustrated) may be inserted into the second aligning apertures 52/152/252/352/452 or the first aligning apertures 42/142/242/342/442 to line up with the second nail apertures 18/118/218/318/418 or the first nail apertures 16/116/216/316/416 of the fibular nail 10/110/210/310. A cavity may then be drilled through the inserted drill guide (not illustrated), through the fibula 2, and possibly through the interosseus membrane (not illustrated) and into the tibia 3 (FIGS. 1 and 2) as well. Screw(s) may then be placed into the first nail apertures 16/116/216/316/416 of the fibular nail 10/110/210/310 or the second aligning apertures 52/152/252/352/452 of the fibular nail 10/110/210/310. Some of the second aligning apertures 52/152/252/352/452 or first aligning apertures 42/142/242/342/442 may be configured to receive resorbable screws, while another plurality of the second aligning apertures 52/152/252/352/452 or first aligning apertures 42/142/242/342/442 may be configured to receive other types of surgical screws (e.g., non-resorbable screws), while another plurality of the second aligning apertures 42/142/242/342/442 may be configured to receive suture buttons. In an embodiment (for example, embodiments depicted in FIGS. 3 and 6), all of the second aligning apertures 52/152/252/352/452 and the first aligning apertures 42/142/242/342/442 may be configured to receive non-resorbable screws. In another embodiment, all of the second aligning apertures 52/152/252/352/452 may be configured to receive non-resorbable screws, the upper first aligning apertures 42/142/242/342/442 may be configured to receive non-resorbable screws, and the lower first aligning apertures 42/142/242/342/442 may be configured to receive resorbable screws.

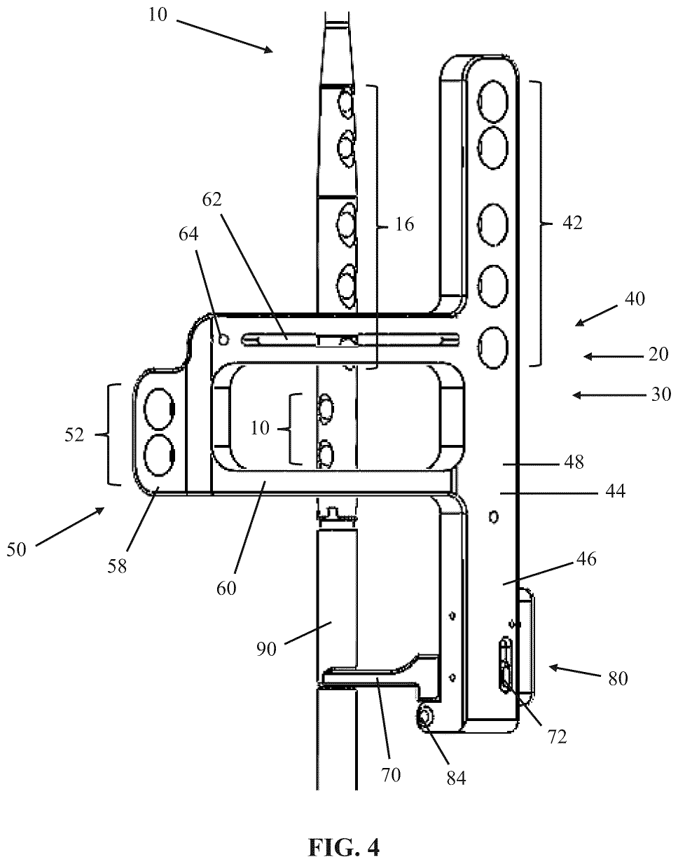

After the fibular nail 10/110/210/310 has been secured in the upper part 4 of the fibula 2 (FIG. 2) and the lower part 6 of the fibula 2 (FIG. 2) with screws, wires, and/or suture buttons, the outrigger 20/120/220/320/420 may be released from its alignment position shown in (for example, the alignment positions shown in FIG. 4 and FIG. 6), to its unobstructed position shown (for example, the unobstructed position shown in FIG. 5), by pressing/clicking/flipping/inserting/screwing/etc. the button 186/286 or the turn knob 487 or thumb screw 292 or key 394 (FIGS. 9, 10, 18, 19, 20, and 22) of releasably locking mechanism 80/180/280/380/480 or any similarly capable mechanism. A surgeon may then manually rotate the outrigger 20/120/220/320/420 from its alignment position (for example, the alignment position shown in FIG. 4 and FIG. 6) to its unobstructed position (for example, the unobstructed position shown in FIG. 5). The outrigger 20/120/220/320/420 may also move automatically, i.e., through a spring or a motor, after a subsequent activation of the releasably locking mechanism 80/180.

With the outrigger 20/120/220/320/420 in its unobstructed position, a surgeon is free to pull, twist, or otherwise manipulate the position and parts of the fibula 2 (FIG. 1 and FIG. 2) so that the bones are in proper relation to each other and in proper position for healing.

The outrigger 20/120/220/320/420 may be capable of being manually manipulated back to its alignment position and locking back into the alignment place automatically. The outrigger 20/120 may also move back to its alignment position automatically with a subsequent activation of the releasably locking mechanism 80/180.

The outrigger 20/120/220/320/420 may be capable of being removed from the fibular nail 10/110/210/310 through multiple means, such as pulling, rotating an element at the distal end of the nail attachment portion 90/190/290/390/490 that unlocks the connection between the fibular nail 10/110/210/310 and the nail attachment portion 90/190/290/390/490, or any other means capable of removing the connection between the fibular nail 10/110/210/310 and the nail attachment portion 90/190/290/390/490.

After the screws and/or wires have been inserted into the fibular nail 10/110/210/310 and the surgery has been positively evaluated, the nail attachment portion 90/190/290/390/490 may be detached from the fibular nail 10/110/210/310, and the main outrigger body 30/130/230/330/430 and the attachment flange 70/170/270/370/470 may also be removed, along with the detachment of the nail attachment portion 90/190/290/390/490. After the outrigger 20/120/220/320/420 has been removed from the fibular nail 10/110/210/310, the internal fixation of the fibula 2 (FIG. 1) may allow the fibula 2 (FIG. 1) to heal. Several progressions of gradually increased loading to the leg may be used in order to avoid/limit weight bearing to the fibula 2 (FIG. 1), such as a cast, a walking boot, a walking shoe, a pair of crutches, a knee scooter, a cane, etc.

While an embodiment of the order of the use of the outrigger 20/120/220/320/420 with the fibular nail 10/110/210/310 has been described in the previous paragraphs, the steps may be reordered, repeated, and omitted as needed. Other steps may also be added, as known to those with ordinary skill in the art.

Additionally, while some examples above illustrate the use of a single wire slot 62/162/462 and a single wire aperture 64/164, alternate configurations of the present invention may encompass multiple wire slots and/or multiple wire apertures in multiple configurations for receiving the multiple Kirschner wires 124 to hold length for and hold the position of the fibula 2 and/or parts of the fibula 2 (e.g., upper part 4 of the fibula 2 and lower part 6 of the fibula 2).

The materials used for the outrigger 20/120/220/320/420 may be stainless steel, titanium, titanium alloy, plastic, carbon fiber, and/or other similar materials.

Referring to FIG. 5, although the outrigger 20 is shown in the unobstructed position at a specific angle (e.g., at about a ninety degrees outwards rotation relative to the aligned position), the main outrigger body 30 may be released in multiple different positions, configurations, and angles to allow a surgeon to have unobstructed visual and physical access to the fractured area (e.g., the main outrigger body 30 may be rotated greater than ninety degrees; in some alternative configurations of the present invention, the main outrigger body 30 may be capable of rotating right or left as well). Additionally, while the releasably locking mechanism 80 is depicted in FIG. 5, any other mechanism that can allow a rotation or movement of the main outrigger body 30 after being locked in an alignment position depicted in FIG. 6 may be used (e.g., the releasably locking mechanism 180 depicted in FIG. 10, the releasably locking mechanism 480 depicted in FIG. 18, the releasably locking mechanism 280 depicted in FIG. 21, and the releasably locking mechanism 380 depicted in FIG. 23). It should be noted that the relative positions of a main outrigger body 30 from a fibular nail 10 in an aligned and unobstructed position described in this paragraph are applicable to all of the embodiments of the present application, (e.g., the embodiments shown in FIGS. 6, 18, 20, and 22).

Pertaining to the screw apertures, such as the first aligning apertures/the first nail apertures 42/16/142/116/242/216/342/316/442/416 and the second aligning apertures/the second nail apertures 52/18/152/118/252/218/352/318/452/418, while some apertures have been described with specific orientations (i.e., lateral to medial and anterior to posterior), the screw apertures may also face in any other orientation to allow for better fixing of the fibular nail 10/110/210/310 to the fibula 2. While the screw apertures are demonstrated in the figures mentioned above as being approximately perpendicular, the screw apertures do not necessarily have to be perpendicular and can align at multiple angles relative to each other for the same reason noted above. The screw apertures have also been described and illustrated with a specific number of apertures; however, any number of apertures may be used.

Also, while releasably locking mechanism 80/180/280/380/480 has been described above as located on the bottom end 46/146/246/346/446 of the first portion 40/140/240/340/440, releasably locking mechanism 80/180/280/380/480 may also be located on several locations on the first portion 40/140/240/340/440 or the attachment flange 70/170/270/370/470.

While screws have been described above in use with apertures, such as the first aligning apertures/the first nail apertures 42/16/142/116/242/216/342/316/442/416 and the second aligning apertures/the second nail apertures 52/18/152/118/252/218/352/318/452/418, any type of screw or fastener may be used. For example, a surgeon may use pedicle screws, cortical screws, cancellous screws, cannulated screws, locking screws, lag screws, self-tapping screws, etc. While Kirschner wires, such as the Kirschner wires 124, have been described above in use with wire slots and wire apertures, such as wire slot 62/162/462 and wire apertures 64/164, other types of stabilization devices may be used such as suture buttons and/or other surgical wires.

Additionally, while certain features may have been described in one or multiple embodiments, each feature may be combined in any embodiment of the present invention. For example, though not illustrated, incision apertures (e.g., incision apertures 264/364) may be located on the screw apertures (e.g., first aligning apertures 42/142/342/442 and second aligning apertures 52/152/452). Another example is that any releasably locking mechanism 80/180/280/380/480 may be located on any outrigger 20/120/220/320/420.

It is to be understood that the above description is intended to be illustrative, and not restrictive. Numerous changes and modifications may be made herein by one of ordinary skill in the art without departing from the general spirit and scope of the disclosure as defined by the following claims and the equivalents thereof. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “have” (and any form of have, such as “has”, and “having”), “include” (and any form of include, such as “includes” and “including”), and “contain” (and any form of contain, such as “contains” and “containing”) are open-ended linking verbs. As a result, a method or device that “has,” “includes,” or “contains” one or more steps or elements possesses those one or more steps or elements but is not limited to possessing only those one or more steps or elements. Likewise, a step of a method or an element of a device that “has,” “includes,” or “contains” one or more features possesses those one or more features but is not limited to possessing only those one or more features. Furthermore, a device or structure that is configured in a certain way is configured in at least that way but may also be configured in ways that are not listed.

It is to be understood that not necessarily all such objects or advantages described above may be achieved in accordance with any particular embodiment. Thus, for example, those skilled in the art will recognize that the systems and techniques described herein may be embodied or carried out in a manner that achieves or optimizes one advantage or group of advantages as taught herein without necessarily achieving other objects or advantages as may be taught or suggested herein.

While the present disclosure has been described in detail in connection with only a limited number of embodiments, it should be readily understood that the present disclosure is not limited to such disclosed embodiments. Rather, the invention can be modified to incorporate any number of variations, alterations, substitutions or equivalent arrangements not heretofore described, but which are commensurate with the spirit and scope of the present disclosure. Additionally, while various embodiments of the present disclosure have been described, it is to be understood that aspects of the disclosure may include only some of the described embodiments.

Components, aspects, features, configurations, arrangements, uses and the like described, illustrated or otherwise disclosed herein with respect to any particular embodiment may be similarly applied to any other embodiment disclosed herein. Accordingly, the present disclosure is not to be seen as limited by the foregoing description but is only limited by the scope of the appended claims.

This written description uses examples to disclose the present disclosure, including the best mode, and also to enable any person skilled in the art to practice the present disclosure, including making and using any devices or systems and performing any incorporated methods. The patentable scope of the present disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1) An outrigger for use in aiding the insertion of an intramedullary nail, comprising:
a nail connecting portion releasably connectable to a distal end of an intramedullary nail;
a main outrigger body connected to the nail connecting portion;
the main outrigger body comprising:
a first portion with a first aligning aperture;
a second portion with a second aligning aperture;
a connecting flange that connects the first portion and the second portion; and
a mechanism for releasably locking the outrigger in an aligned position with the intramedullary nail, wherein the aligned position comprises the first aligning aperture in concentric alignment with a first nail aperture of the intramedullary nail to allow an alignment of a fastener with the first nail aperture through the first aligning aperture.

2) The outrigger of claim 1, wherein the mechanism is configured to allow movement of the outrigger to an unobstructed position with the intramedullary nail, the outrigger releasably lockable in the unobstructed position and the first aperture non-concentrically aligned with the first nail aperture of the intramedullary nail.

3) The outrigger of claim 1, wherein:
the first portion has a first face;
the second portion has a second face;
the first face approximately perpendicular to the second face.

4) The outrigger of claim 1, wherein:
the connecting flange further comprises a wire aperture;
the wire aperture configured to hold an insertion of a surgical wire therethrough when the outrigger is in the aligned position.

5) The outrigger of claim 1, wherein:
the connecting flange further comprises a wire slot;
the wire slot extending between the first portion and the second portion; and
the wire slot configured to confine a translation of a surgical wire within a longitudinal axis therethrough when the outrigger is in the aligned position.

6) The outrigger of claim 1, wherein the mechanism comprises:
a locking mechanism that retains a position between the attachment flange and the main outrigger body;
a rotation element coupled to the main outrigger body, the rotation element rotatably coupling the main outrigger body around an axis of the rotation element; and
an actuator connected to the locking mechanism, the actuator capable of disengaging the locking mechanism such that that the main outrigger body can rotate around the axis of the rotation element.

7) The outrigger of claim 3, wherein:
the first face has a first longitudinal axis;
the second face has a second longitudinal axis;
the intramedullary nail has a third longitudinal axis;
the first longitudinal axis is parallel to the third longitudinal axis; and
the second longitudinal axis is parallel to the third longitudinal axis.

8) The outrigger of claim 5, wherein the locking mechanism further comprises:
a cavity within the main outrigger body;
at least one spring coupled to an end of the cavity;
a button coupled to an end of the at least one spring; and
a retention element coupled to the at least one spring and the button to keep the at least one spring and the button at least partially within the cavity.

9) The outrigger of claim 5, wherein the locking mechanism further comprises:
a first hole coupled to the attachment flange;
a second hole coupled to the main outrigger body; and
a fastener.

10) The outrigger of claim 8, wherein:
the first hole is threaded;
the second hole is threaded; and
the fastener comprises a screw.

11) The outrigger of claim 1, wherein the intramedullary nail is a fibular nail.

12) A method of internal fixation, comprising:
inserting an intramedullary nail into a bone;
attaching an outrigger to the intramedullary nail;
concentrically aligning a first nail aperture of the intramedullary nail with a first aligning aperture of the outrigger;
fastening a first fastener into the first nail aperture by inserting the first fastener through the first aligning aperture;
positioning a first portion of the bone relative to a second portion of the bone;
inserting at least one surgical wire through a wire aperture on the outrigger to hold the first portion of the bone or the second portion of the bone in place;
fastening a second fastener into a second nail aperture of the intramedullary nail by inserting the second fastener through a second aligning aperture of the outrigger; and
removing an outrigger from the nail connecting portion.

13) The method of claim 12, wherein the positioning the first portion of the bone relative to the second portion of the bone further comprises rotating the outrigger.

14) The method of claim 12, wherein the positioning the first portion of the bone relative to the second portion of the bone further comprises:
inserting a surgical wire through a wire slot on the outrigger into the first portion of the bone or the second portion of the bone; and
rotating the surgical wire to rotate the first portion of the bone or the second portion of the bone.

15) The method of claim 12, further comprising fastening a third fastener into a third nail aperture of the intramedullary nail by inserting the third fastener through a third nail aperture of the outrigger.

16) The method of claim 12, further comprising:
unlocking a releasably locking mechanism of the outrigger;
rotating the outrigger into an unobstructed position;
moving the first portion of the bone relative to the second portion of the bone;
rotating the outrigger into an alignment position; and
locking the releasably locking mechanism of the outrigger.

17) The method of claim 12, further comprising removing the at least one surgical wire from the bone.

18) The method of claim 16, wherein rotating the outrigger into an unobstructed position is actuated by pushing a button.

19) The method of claim 16, wherein rotating the outrigger into an unobstructed position is actuated by turning a knob.

20) The method of claim 16, wherein rotating the outrigger into an unobstructed position is actuated by rotating a thumb screw.

21) The method of claim 16, wherein rotating the outrigger into an unobstructed position is actuated by removing a key.
